(19) United States
(12) Patent Application Publication
(10) Pub. No.: US 2006/0233705 A1
(43) Pub. Date: Oct. 19, 2006

(54) Diagnosis by determination of hyperactivity or increased expression of members of cell signaling pathways

(76) Inventors: Hildegard M. Schuller, Knoxville, TN (US); George W. Kabalka, Knoxville, TN (US)

Correspondence Address: HOWARD EISENBERG, ESQ., 2206 APPLEWOOD COURT, PERKASIE, PA, 18944, US

(21) Appl. No.: 11/109,428

(22) Filed: Apr. 19, 2005

(51) Int. Cl. A61K 51/00; G01N 33/574

(52) U.S. Cl. 424/1.49; 435/7.23

(57) ABSTRACT

A non-invasive method for determining the presence or severity of a bodily disorder associated with hyperactivity or increased expression of a signal transduction protein, transcription factor, or protein kinase that is a member of the MAPK or GPCR pathways. A reagent that binds to such a signal transduction protein, transcription factor, or protein kinase is non-invasively contacted to a tissue or fluid within the body of a subject or to a fluid removed from a subject, the reagent is permitted to bind to the signal transduction protein, transcription factor, or protein kinase in the tissue or fluid, the presence of binding of the reagent to the signal transduction protein, transcription factor, or protein kinase in said tissue or fluid is determined, and the binding is correlated with the presence or severity of said bodily disorder within the subject.

### GOVERNMENT FUNDING

It is hereby acknowledged that the U.S. Government has certain rights in the invention described herein, which was supported in part by grants R01CA096128, R01CA42829, and R01CA088809 from the National Institutes of Health (NIH).

### FIELD OF THE INVENTION

The invention pertains to the field of diagnosis of disease. More particularly, the invention pertains to the field of diagnosis of diseases associated with hyperactivity or increased expression of signal transduction proteins, transcription factors, and/or protein kinases.

### BACKGROUND OF THE INVENTION

Mammalian cells contain a wide array of receptors on their surface that provide mechanisms for transduction of signals that are external to the cell to an effector protein or transcription factor within the cellular cytoplasm or nucleus. When a cell surface receptor interacts with a signal, the receptor undergoes a change that permits its intracellular portion to interact with a protein inside the cell. Often this internal protein will undergo a change, such as phosphorylation, to become activated and, in this activated state, will interact with and will directly or indirectly activate another intracellular protein. This second activated protein may activate one or more additional proteins in a cascade until a final effector protein is activated which causes the cell to respond to the signal.

One widely studied signal-induced protein activation cascade is the Ras-Raf-MEK-ERK pathway, also known as the MAPK/ERK pathway, the MAPK pathway, or the ERK pathway. (MEK=MAPK/ERK kinase, ERK=extracellular-signal-regulated kinase, MAPK=mitogen-activated protein kinase). This protein cascade is initiated when an external chemical signal, such as epidermal growth factor (EGF) induces dimerization of a surface receptor, such as EGF receptors (EGFR), which causes autophosphorylation and activation of tyrosine kinases (p-tyrk). The p-tyrk elicits a conformation change in Ras which enables it to bind and activate Raf Raf in turn phosphorylates and activates MEK which in turn activates ERK. Activated ERK has many substrates within the cell upon which it acts.

This cascade has been extensively studied because several of the genes that encode the proteins of this cascade are proto-oncogenes. Proto-oncogenes are genes that encode proteins that stimulate or enhance the division and/or viability of cells and which, when mutated or otherwise damaged, may lead to uncontrolled growth or division of cells and thus to cancer. Such mutated or otherwise damaged proto-oncogenes are referred to as oncogenes. The genes encoding EGFR, Ras, and Raf have been shown to be proto-oncogenes, mutations of which lead to hyperactivity of the mutated protein and increased expression of proteins downstream in the cascade from the mutated protein.

Many types of cancers are associated with hyperactivity and/or increased expression of proteins of the MAPK pathway. Such cancers include those of the breast, colon, pancreas, ovary, prostate, skin, lung, thyroid, and central nervous system.

Because of the association with cancer of hyperactivity/increased expression of the proteins in this pathway, several clinical trials have been initiated to attempt to treat cancer by inhibiting these proteins, particularly Ras and Raf and EGFR-specific tyrosine kinases. To date, although such clinical trials have produced some positive results, overall the clinical results have been disappointing.

Another important signal-induced protein activation pathway is the G protein-coupled receptor (GPCR) pathway. This pathway is initiated when a signal at the cell surface in the form of a ligand binds to the extracellular portion of a G protein-coupled receptor on the surface of a cell. The ligand may be a variety of signals, such as a corticotrophin, dopamine, epinephrine or other beta-adrenergic agonist, follicle stimulating hormone, glucagons, or a prostaglandin. The binding of the ligand to the GPCR results in a conformation change in the receptor which further results in the binding of intracellular domain of the GPCR to an intracellular G protein, so named because it binds GDP/GTP. The G protein then interacts with and activates adenylate cyclase which converts ATP into cyclic AMP (cAMP). cAMP then activates protein kinase A (PKA) which phosphorylates other proteins and results in the activation of the intranuclear transcription factor CREB (cAMP response element binding protein). The activated CREB turns on gene transcription, causing the cell to produce the appropriate gene products in response to the signal at the cell surface. PKA can also activate phospholipase-A2 (PLAP-2) which releases arachidonic acid (AA) from cell membrane phospholipids. AA, as well as its metabolites, can activate transcription factors to induce cell proliferation.

The binding of the GPCR to the intracellular G protein may also result in the release of a messenger compound, diacylglycerol (DAG). DAG then binds to and activates protein kinase C (PKC), which in turn activates Raf, thus forming a linkage between the MAPK and the GPCR pathways.

Recently, it has been discovered that beta-adrenergic agonists, such as isoproterenol and the nicotine derived nitrosamine 4-(methylnitrosamino)-1-(3-pyridyl)-1-butanone (NNK), reproducibly stimulate the proliferation of human pulmonary adenocarcinoma cells in vitro by increasing the release of arachidonic acid. Schuller, H M et al, Cancer Research 59, 4510-4515 (1999). In an animal model, NNK reproducibly induced pulmonary adenocarcinomas and the development of these tumors was promoted by beta-adrenergic agonists whereas a beta-blocker prevented the development of these tumors. Schuller, H M, et al, J. Cancer Res. Clin. Oncol. 126:624-630 (2000). Thus, the GPCR cascade, like the MAPK cascade, is associated with the development of cancer. Unlike the MAPK cascade, the inventors are not aware of any clinical trials that have been initiated to determine the efficacy of inhibition of the GPCR cascade in the treatment of cancer.

In addition to cancer, hyperactivity or increased expression of members of either or both of the MAPK pathway and of the GPCR pathway occurs in other disease conditions, such as cardiovascular and neurologic diseases.

It is well established that, in general, if a diagnosis of cancer can be made in its earliest stages, preferably before a patient is suffering from symptoms of the disease, there will be a more favorable prognosis for that patient. For this reason, several screening tests have been developed to detect cancer in its earliest stages. For example, the detection of fecal blood is an indication of the possible presence of a gastrointestinal cancer. Mammography and self-examination are used to detect breast cancer in its early stages. Digital palpation and determination of the level of prostate specific antigen (PSA) are used for early detection of prostate cancer. And PAP smears are used for early detection of cancer of the cervix.

These tests, although highly efficacious, have several disadvantages. Each of these tests is associated with a certain degree of false negative and false positive results, as is the case for any diagnostic test. More importantly, these tests are specific for cancers occurring at a particular site in the body. That is, these tests cannot be used as a general screen for cancer within a patient. And of course, these tests cannot be used to determine the site of cancer in a patient who is suffering from a cancer of unknown origin.

Additionally, for most cancers, such as cancers of the pancreas, lung, ovary, central nervous system, liver, and upper urinary tract, there are no early screening procedures that are presently available. With these types of cancer, a diagnosis of cancer is available only after a patient has begun to experience symptoms due to the cancer. At that time, the cancer will be in a more advanced stage and the prognosis for the patient will be less favorable.

A significant need exists for a method of diagnosis of cancer that is independent of the site or type of cancer and which is capable of providing an early diagnosis, preferably before a patient is suffering from symptoms of the disease. A significant need exists for a method of diagnosis of the site of cancer in a patient who is suffering from a cancer of unknown origin. Preferably, such a test should be a non-invasive test, one that does not require the removal of a solid tissue from an individual.

### BRIEF DESCRIPTION OF THE FIGURES

FIG. 1 shows a simplified schematic diagram of the G Protein-Coupled Receptor (GPCR) pathway and the Mitogen-Activated Protein Kinase (MAPK) pathway, and the interaction of these two pathways.

### DESCRIPTION OF THE INVENTION

It has recently been discovered that many cancers and other bodily disorders are associated with hyperactivity and/or overexpression of the MAPK pathway. It has also recently been discovered that many cancers are associated with hyperactivity and/or overexpression of the GPCR pathway. The inventors have further discovered that the hyperactivity and/or overexpression of these two pathways occurs simultaneously in many cancers and that agonist binding of the G-protein coupled receptor may cause transactivation of the MAPK pathway.

In one embodiment, the invention is a non-invasive method for determining the presence or severity of a bodily disorder associated with hyperactivity or increased expression of a signal transduction protein, transcription factor, or protein kinase that is a member of the MAPK or GPCR pathways. According to this embodiment of the invention, a reagent that binds to such a signal transduction protein, transcription factor, or protein kinase is non-invasively contacted to a tissue or fluid within the body of said subject or to a fluid removed from said subject, the reagent is permitted to bind to the signal transduction protein, transcription factor, or protein kinase in the tissue or fluid, the presence of binding of the reagent to the signal transduction protein, transcription factor, or protein kinase in said tissue or fluid is determined, and the binding is correlated with the presence or severity of said bodily disorder within said subject.

As used herein, the term “non-invasive” refers to a test or a method that does not utilize the opening of a body cavity of a subject or removal of a solid tissue sample from the body of a subject. Blood cells or other cells contained within fluid removed from a subject are not considered to be solid tissue samples and thus the removal of a blood sample or other fluid sample, such as urine, CSF fluid, saliva, semen, fluid aspirates from an organ or body cavity, and transudates or exudates may be included as part of a non-invasive method according to the invention. The term “non-invasive” is used herein to specifically exclude surgically removed solid tissue, such as by biopsy or necropsy, from the invention.

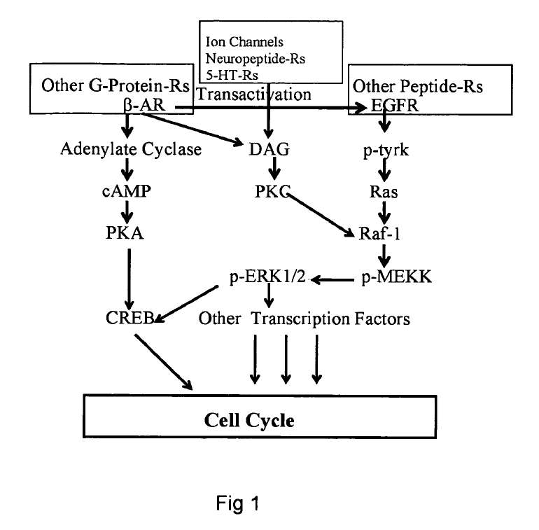

The term “member” of the MAPK pathway or of the GPCR pathway is used herein as a generic term to refer to a signal transduction protein, transcription factor, or protein kinase that is included in such pathway. FIG. 1 shows a simplified flow diagram of these two pathways and their interconnection. As shown in FIG. 1, members of the MAPK pathway include, but are not limited to, EGFR, Ras, Raf, MEK, ERK, and protein kinases, such as tyrosine kinases, upstream of Ras, such as phosphatidylinositol 3-kinase. FIG. 1 also shows that members of the GPCR pathway include, but are not limited to, GPCR, adenylate cyclase, cAMP, PKA, CREB, DAG, and PKC. Both the activated, such as phosphorylated, and the inactivated forms of such members are included.

The term “reagent” refers to a chemical entity that binds to a member of the MAPK pathway or to a member of the GPCR pathway or to members of both pathways. Included within the term “reagent” are antibodies, such as monoclonal or polyclonal antibodies, and non-antibody chemical compounds, including small molecules and polymers, such as polypeptides. Such reagents may or may not be capable of inhibiting the activity of a member of either of these pathways.

In accordance with the method of the invention, the presence or absence of hyperactivity or overexpression of one or more members of the MAPK pathway or of the GPCR pathway may be determined. In a preferred embodiment, the hyperactivity or overexpression of one or more members of each of the MAPK pathway and the GPCR pathway is determined. The determination of the presence or absence of overexpression or hyperactivity of both of these pathways may be advantageous in particular situations.

The method of the invention may be used diagnostically in several manners. The method of the invention may be used as a non-invasive screening test to identify individuals who may be suffering from a disease associated with hyperactivity or overexpression of a member of the MAPK or GPCR pathways. Such diseases include, for example, cardiovascular and neurologic diseases and various cancers.

The method of the invention for use as a screening test is preferably performed as an in vitro method by obtaining a fluid sample, preferably a blood sample, from a subject. The sample may be obtained from the subject by any applicable method such as by venipuncture, needle aspiration of an organ or body cavity, catheterization such as of the intestinal, genital, or urinary tracts, from sputum samples, from epithelial swipes obtained by procedures such as bronchoscopic brushing or vaginal smears, or collection of eliminated fluid, such as urine, pus, saliva, tears, vaginal secretions, feces, nasal or ocular discharge, or vomitus. Preferably, the sample for screening is a blood sample obtained by venipuncture.

The sample thus obtained is contacted, and preferably mixed, with a reagent that binds to a member of the MAPK and/or of the GPCR pathways. The reagent may be an antibody, such as a monoclonal or polyclonal antibody. Alternatively, the reagent may be a chemical compound other than an antibody. The reagent may or may not have a chemical or biological effect on the member. That is, the reagent may be an inhibitor of the member, although whether or not the reagent inhibits a member of either pathway is immaterial.

Antibodies that bind to the phosphorylated or unphosphorylated forms of the various members of the MAPK pathway or the GPCR pathway are obtainable from commercial sources. Alternatively, such antibodies may be obtained by standard methods for producing antibodies, such as by the use of hybridoma techniques.

Chemical compounds other than antibodies that bind to several of the members of the two pathways are known. For example, the compound BAY 43-9006 (Sorafenib, Onyx Pharmaceuticals, Richmond, Calif., USA) binds to the Raf protein and inhibits its ability to activate MEK. Another inhibitor of Raf is ZM 336372 (3-(Dimethylamino)-N-[3-[(4-hydroxybenzoyl)-amino]-4-methylphenyl]benzamide) (AstraZeneca Pharmaceuticals LP, Wilmington, Del., USA). Another example is the MEK inhibitor CI-1040 (Pfizer, Ann Arbor, Mich., USA). Another MEK inhibitor is PD-0325901 (Pfizer). Another MEK inhibitor is PD 98059 (2′-amino-3′-methoxyflavone, LC Laboratories, Woburn, Mass., USA). An example of an inhibitor of PKA is 4-cyano-3-methylisoquinoline (EMD Biosciences, Inc., Madison, Wis., USA). An example of an adenylate cyclase inhibitor is 9-(tetrahydro-2-furanyl)-H-6-amine (Calbiochem, La Jolla, Calif., USA). Additional inhibitor compounds that bind to members of the MAPK pathway or of the GPCR pathway are available commercially, for example from EMD Biosciences, Inc.

After contacting the reagent with the sample, it is determined if the reagent has bound to a member. The sample may be treated to remove any unbound reagent, such as by rinsing, centrifugation, or filtering. The determination of binding may be by any method that provides such determination. In a preferred embodiment, the reagent is labeled to facilitate detection of a reagent/member complex and thus a determination of binding. Examples of suitable labels include biotin labels, chemiluminescent labels, radioisotope labels, and fluorescent labels.

Preferably, the amount of binding of the reagent to the sample is compared to the amount of binding found in similar samples taken from individuals that are not suffering from a bodily disorder associated with increased expression or hyperactivity of the MAPK or GPCR pathways. Preferably, the comparison is to a historical control indicating the normal level of binding. In such a way, the amount of binding of the reagent to the sample may be correlated to the presence or absence of a bodily disorder associated with such increased expression or hyperactivity, and thus serve as a means of diagnosing or of aiding in the diagnosis of such a bodily disorder.

The presence and the amount of binding may be determined by any method by which such determinations may be made. For example, binding may be determined visually, for example by means of a chemiluminescent label that is utilized as a component in an ELISA test. The determination may also be made by flow cytometry, a microarray technology using a panel of multiple antibodies, or other means capable of providing quantitative information.

The screening method may be used to non-specifically determine that a bodily disorder exists in a subject. For example, the screening method may be performed by collecting a sample, such as a venous blood sample, from a subject who is presently not experiencing symptoms of a bodily disorder, such as cancer or cardiovascular disease, or who is experiencing symptoms that are not specific to any particular type of bodily disorder. A positive result, binding of the reagent to the sample at a level that is higher than normal, will provide an alert that a bodily disorder exists and that additional testing is necessary to more definitively diagnose the disorder. Such additional testing may include the in vivo method of the invention described below.

The in vitro method may also be used when it is known that a patient is suffering from a particular disorder, such as cancer or a cardiovascular disease. The method may be used as an aid in determining the progression of a bodily disorder, the response of a bodily disorder to therapy, or whether a particular type of therapeutic intervention is warranted to treat the bodily disorder in the subject.

For example, in the case of a subject suffering from a known cancer, the method may be used to determine whether treatment should be initiated with a therapeutic medication that inhibits the MAPK pathway or the GPCR pathway or both of these pathways. If the test method of the invention provides an assessment that a member of one or both of these pathways is hyperactive or overexpressed, then treatment with an inhibitory medication may be indicated. Alternatively, if the method of the invention provides an assessment that there is no hyperactivity or overexpression of either of these pathways, then such inhibitory therapy would most likely not be indicated or may even be harmful.

As another example, in the case of a subject suffering from a disorder such as a cancer and being treated with a medication that inhibits either or both of the pathways, the in vitro method of the invention may be used to monitor response to such therapy. If, for example, the amount of binding significantly decreases following such therapy, such result indicates a positive response to the therapy. In contrast, a finding of no decrease or of an increase in binding following the initiation of therapy would indicate a lack of response and that a change in therapeutic intervention may be warranted.

When testing for presence of overexpression or hyperactivity of the MAPK pathway, a preferred reagent to be used is one that binds to the Raf protein. As shown in FIG. 1, the Raf protein may be activated by Ras or may be activated by PKC as a consequence of agonist binding of the G-protein coupled receptor. Because both binding of the GPCR and of the EGF receptor can cause increased activity of the Raf protein, it is preferred that this protein, or a protein downstream from Raf such as the MEK or ERK protein, be the subject of the test method of the invention.

When performing the test of the invention, it is preferred that hyperactivity and/or overexpression of both the two pathways be determined. Recently, there have been several clinical trials of chemical agents that inhibit the Raf protein for use as antineoplastic agents. Results of these trials have been disappointing. It is conceived by the inventors that the failure of these clinical trials may have been due, at least in part, to the fact that in many cancers there is simultaneous hyperactivity and/or overexpression of both of the pathways. As shown in diagram of FIG. 1, each of the two pathways can result ultimately in hyperactivity of CREB. Because CREB is hyperactivated, not only indirectly by Raf, but also by PKA, the inhibition of the Raf protein fails to remove the hyperactivation of CREB, which continues even though the MAPK pathway has been inhibited.

Another way in which the method of the invention may be practiced is as an in vivo method to diagnose or aid in the diagnosis of a bodily disorder that is associated with hyperactivity and/or overexpression of the MAPK and/or GPCR pathways. According to the in vivo method of this embodiment of the invention, a labeled reagent that binds to a signal transduction protein, transcription factor, or protein kinase which is a member of the MAPK pathway or the GPCR pathway is administered to a mammalian subject and is permitted to be distributed within the body of the subject, the reagent is permitted to bind to the member of the MAPK or GPCR pathways within the body of the subject, and the presence of one or more areas within the body of the subject where the labeled reagent has bound is determined. The existence of localized concentration of binding of the labeled reagent within the body establishes the presence of localized elevated expression of the particular member of the MAPK or GPCR pathway to which the reagent binds or of hyperactivity of all or a portion of the pathway that includes that member.

This information so obtained, either alone or with other clinical or laboratory information, permits the diagnosis of a disorder associated with overexpression or hyperactivity of a member of the MAPK and/or GPCR pathways.

The method of the invention may also be used to monitor the progression of a disease process associated with such overexpression or hyperactivity, and to determine, for example, if the disease process is progressing or is being ameliorated over time. As with the in vitro method described above, the in vivo method may also be used to determine response to therapy or to determine if a particular therapy is indicated for a particular individual suffering from a bodily disorder.

The particular reagent that binds to a member of the MAPK and/or of the GPCR pathways that is used in the in vivo diagnostic method of the invention is immaterial, although it is preferred that such reagent does not have a deleterious physical effect on the subject to whom it is administered. The reagent may be an antibody, such as a monoclonal or polyclonal antibody. Alternatively, the reagent may be a chemical compound other than an antibody. The reagent may or may not have a chemical or biological effect on the member. That is, the chemical compound may be an inhibitor of the member, although whether or not the chemical compound inhibits the member is immaterial.

Antibodies and chemical compounds that bind to the various members of the MAPK pathway or to the various members of the GPCR pathway are described above. Such antibodies and chemical compounds may be synthesized or may be purchased commercially.

The label may be any label that can be attached to an antibody or chemical compound and, when such labeled antibody or chemical compound is administered to a mammalian subject, can be detected within the body of the subject by non-invasive means, that is by means other than detecting the presence of the label on or in a solid tissue sample that has been removed from the subject. Examples of such labels include biotin labels, chemiluminescent labels, radioisotope labels, fluorescent labels, and nuclear magnetic resonance labels. Radioisotope labels are preferred.

The method by which the presence of label bound to a member of either or both of the MAPK and GPCR pathways within the body of the patient is determined may vary depending on several factors, including route of administration of the labeled reagent, location within the body where the label is concentrated, and type of label. For example, visual inspection may be the appropriate method for determination of the presence of a fluorescent or chemiluminescent label that is bound to a member of these pathways in the skin or within the eye.

In a preferred embodiment, the presence of label bound to a member of these pathways is determined by an imaging modality that produces an image of a portion or of the entire body of a subject. Such imaging modalities include magnetic resonance imaging (MRI), radiography such as computerized coaxial tomography (CAT), or nuclear imaging. Nuclear imaging modalities utilize a radioactive label and the presence of the label within the body of a subject is determined by a radiation detector. Examples of suitable nuclear imaging modalities include conventional nuclear medicine, positron emission tomography (PET), and single photon emission computerized tomography (SPECT).

In a most preferred embodiment, the presence of bound label within the body is determined by a computerized nuclear medicine modality, such as PET or SPECT scan, including Micro-PET and Micro-SPECT. Utilizing a radiolabeled derivative of a reagent that binds to a member of either or both of the MAPK or GPCR pathways as a tracer for PET or SPECT scan, the presence of a disease condition associated with hyperactivity or overexpression of the members of these pathways enzyme within a mammalian subject may be diagnosed or monitored.

Tracers, radioactively labeled chemical compounds used for PET or SPECT scanning, that are preferred for the method of the invention include radiolabeled, preferably radiohalogenated, derivatives of reagents that bind to a member of the MAPK or GPCR pathways. Preferred radiohalogenated labels for such tracers include radioactive fluorine, iodine, and bromine, such as ¹⁸F, ⁷⁶Br, ¹²³I, and ¹²⁴I. Labels for such tracers other than radiohalogenated tracers include radioactive nitrogen such as ¹³N, radioactive oxygen such as ¹⁵O, and radioactive carbon such as ¹¹C. Labels of radioactive halogens are preferred because of their relatively long half-lives.

The reagent, such as the antibody or non-antibody chemical compound, may be labeled, such as radiolabeled, by any method by which the label may be adhered or connected to the reagent. Methods for labeling antibodies and chemical compounds, such as biotin labeling, fluorescent labeling, radioisotopically labeling, chemiluminescent labeling, or nuclear magnetic resonance labeling, are well known in the art and may be readily adapted by one skilled in the art to label a reagent in accordance with the invention. Commercial kits, such as those marketed by Pierce Biotechnology, Inc., Rockford, Ill., US, are available that provide for the labeling of chemical compounds, and such kits may be used to label the reagent.

The in vivo method of the invention is useful to determine whether or not a patient has a disease, such as a cancer, or to differentiate patients suffering from a disease that is associated with overexpression and/or hyperactivity of a member of either or both the MAPK and GPCR pathways from those patients that are suffering from the disease but whose disease is not associated with such overexpression and/or hyperactivity. Because not all patients with diseases that are associated with overexpression or hyperactivity of the MAPK and/or GPCR pathways, such as cancers of the lung, breast, prostate, colon, or pancreas, do indeed overexpress or have hyperactive members of these pathways, and because treatment of such individuals with an inhibitor when the disease in these patients is not associated with such overexpression or hyperactivity is typically not beneficial and may in fact be harmful, the method of the invention may be used to determine if a particular patient should or should not be treated for the disease condition with an inhibitor of one or more members of these pathways.

The invention is further illustrated in the non-limiting examples that follow. The examples describe the invention with reference to a Syrian golden hamster model of human cancer that overexpresses members of the MAPK and GPCR pathways and with reference to particular labeled compounds that bind to these members. One skilled in the art will readily comprehend that the invention is applicable to mammals other than hamsters, including humans and domesticated animals such as dogs, cats, and farm animals, to diseases other than the exemplified cancer, to labels other than radioactive labels, to radioactive labels other than radiohalogens, to detection modalities other than nuclear medicine, to nuclear medicine modalities other than PET, and to reagents, including antibodies, that bind to members of the MAPK and/or GPCR pathways, other than those exemplified.

### EXAMPLE 1

Syrian Golden Hamsters are injected subcutaneously with the tobacco-specific carcinogenic nitrosamine 4-(methylnitrosamino)-1-(3-pyridyl)-1-butanone (NNK) at a dosage of 2.5 mg/100 g bodyweight three times weekly for 10 weeks. NNK has been shown to induce cancers of various organs, including lung, pancreas, and liver. Control hamsters are untreated. One week after the final NNK injection, approximately 250 microliters of venous blood is collected from the hamsters.

### EXAMPLE 2

Antibodies that bind to phosphorylated or unphosphorylated members of the MAPK and GPCR pathways, including Raf, Ras, MEK, ERK, adenylate cyclase, cAMP, PKA, CREB, and PKC are purchased from commercial sources, including EMD Biosciences (San Diego, Calif.) (Raf, ERK), BD Biosciences Pharmingen (San Diego, Calif.) (Raf), Cell Signaling Technology (Beverly, Mass.) (Ras), Abgent (San Diego, Calif.) (MERK), GenWay Biotech Inc. (San Diego, Calif.) (adenylate cyclase), Abcam, Inc. (Cambridge, Mass.) (cAMP, PKA, PKC, Raf), and Upstate (Charlottesville, Va.) (CREB).

Paired antibodies for each of the above member of the MAPK and GPCR pathways are utilized as coating antibody and detection antibody in formulating an ELISA (Enzyme-Linked Immunosorbent Assay) for each of the members. ELISA tests for each of the pathway members are developed using the ENDOGEN® ELISA Development Kit from Pierce Biotechnology Inc.

Briefly, a first antibody that binds to a member of the MAPK or GPCR pathway is bound to the wells on a microtiter plate. The plate is washed to remove unbound antibody and a blocking reagent is added to each well to block any well surfaces not bound by antibody. The wells are then washed. A blood sample from the test hamsters of Example 1 is introduced into each well. The wells are then washed again to remove any portion of the sample that has not bound to the antibody. A labeled second antibody that binds to the same protein as the does the first antibody is then added to each well. The plates are then washed to remove any of the second antibody that has not bound. A detection reagent such as TMB/hydrogen peroxide is added, which develops a blue color in proportion to the amount of bound analyte in the wells. Then a stop solution is added to the wells and the color intensity of each well is measured using an ELISA plate reader.

The color intensity of the hamsters that have been treated with NNK is determined to be significantly higher than that of the control hamsters. Histological and histochemical evaluation of the lungs, pancreas, and liver of the hamsters confirms the presence of early premalignant lesions in the pancreas (formation of pseudo-ducts) and highly positive immunoreactivity to an antibody against members of the MAPK and GPCR pathways in these organs of the NNK treated hamsters.

### EXAMPLE 3

Radiohalogenated analogues of BAY 43-9006, an inhibitor of the Raf protein, suitable for use as tracers in PET or SPECT imaging, are produced. The structure of BAY 43-9006 is shown below as Formula 1. Table 1 shows the radiohalogenated analogues of BAY 43-9006, compounds 1a to 1h, that are produced.

TABLE 1
Radiohalogenated BAY 43-9006 Analogues

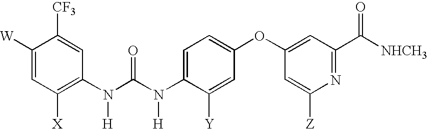

1a: W = ¹⁸F; X = H; Y = H; Z = H
1b: W = Cl; X = ¹⁸F; Y = H; Z = H
1c: W = Cl; X = H; Y = ¹⁸F; Z = H
1d: W = Cl; X = H; Y = H; Z = ¹⁸F
1e: W = ¹²⁴I; X = H; Y = H; Z = H
1f: W = Cl; X = ¹²⁴I; Y = H; Z = H
1g: W = Cl; X = H; Y = ¹²⁴I; Z = H
1h: W = Cl; X = H; Y = H; Z = ¹²⁴I

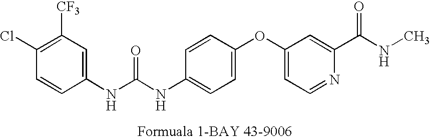

The overall synthetic scheme for preparing the BAY 43-9006 analogues is based on the efficient coupling of labeled bis aryl ethers with anilines utilizing the carbonydiimidazole (CDI) chemistry as shown below in Scheme 1. This coupling reaction is facile, high yield, and tolerates a variety of functional substituents. 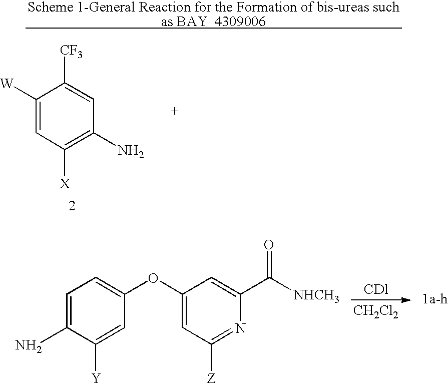
Scheme 1—General Reaction for the Formation of bis-ureas such as BAY 4309006

Generally, to minimize undesired side reactions during the radiohalogenation sequences, radiohalogenated compound 2 of Scheme 1 is prepared and coupled to compound 3 to form product ureas as described below. Radioiodinations are carried out on boronated analogues of compound 1 that are radiohalogenated in the final step, as described below.

### Example 3a

### Synthesis of Fluorine-18 Analogues, Compounds 1a to 1d

These syntheses utilize the preparation of a 4-[¹⁸F]fluoroaniline precursor which is then coupled to diaryl ethers as outlined in the following schemes.

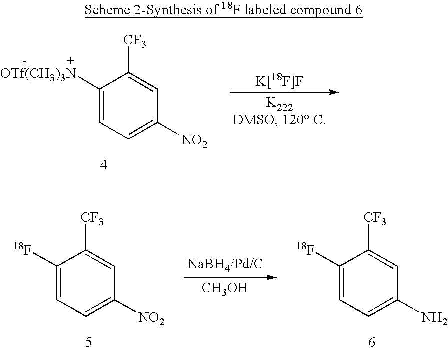

The preparation of compound 6 commences with the formation of the trimethylammonium derivative, compound 4, from the corresponding amine. Formation of compound 5 is than achieved using well known nucleophilic fluorination chemistry. The resultant aniline derivative, compound 6, obtained after reduction with sodium borohydride, is coupled to 4-(4-aminophenoxy)pyridinecarboxylic acid methylamide, compound 7, (prepared by coupling 4-aminophenol to picolonic acid according to literature procedures) as shown in Scheme 3 which shows production of compound 1a.

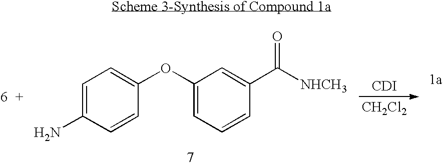

Synthesis of analogue compound 1b is initiated by preparing 4-chloro-2-[¹⁸F]fluoro-5-trifluoromethylaniline, compound 10, from the corresponding nitro derivative, compound 8, as shown in Scheme 4. Intermediate compound 8 is synthesized by exhaustive methylation of the precursor aniline derivative.

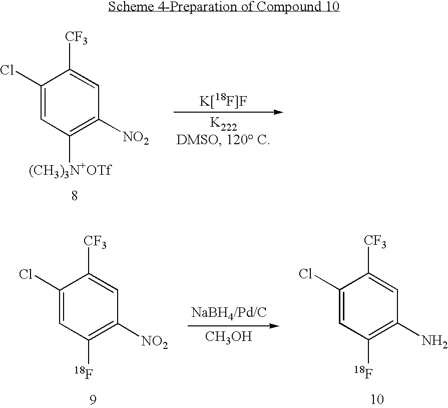

The aniline compound 10 is then coupled to the aryl ether compound 7 shown in Scheme 3 to generate compound 1b, as shown in Scheme 5.

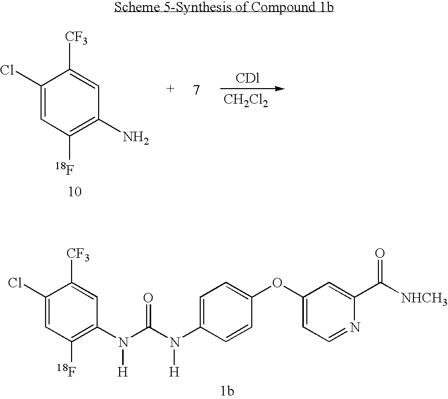

Compound 1c is prepared by synthesizing the corresponding radiohalogenated aryl ether, compound 16, as shown in Scheme 6. This synthesis commences with the preparation of an amino substituted derivative, compound 13, by coupling 4-chloropicolonic acid monomethylamide, 11, with phenol compound 12.

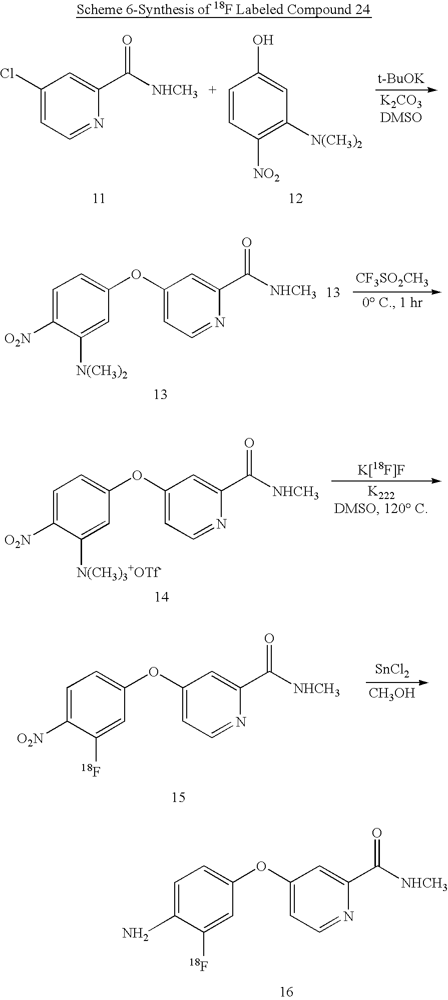

Methylation of compound 13 generates the trimethylammonium intermediate compound 14 which is subjected to nucleophilic radiofluorination to generate compound 15. Intermediate compound 15 is reduced to the corresponding amine, compound 16, utilizing stannous chloride. Analogue compound 1c is prepared by coupling compound 16 with 4-chloro-3-trifluoromethylanaline, compound 17, using the CDI methodology, as shown in Scheme 7.

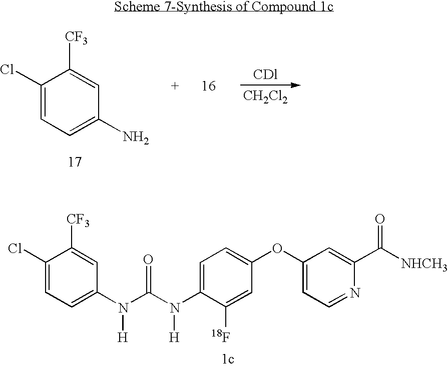

The preparation of analogue compound 1d is as follows. Preparation of compound 22, as shown in Scheme 8, is initiated with the conversion of 4,6-dibromopicolinic acid, compound 18 (prepared by oxidation of the corresponding 4,6-dibromo-2-methylpyridine) to the corresponding monomethylamide compound 19 which is then coupled to p-nitrophenol, 20, to form aryl ether compound 21. Reduction by stannous chloride to 22 followed by nucleophilic fluorination then generates intermediate compound 23.

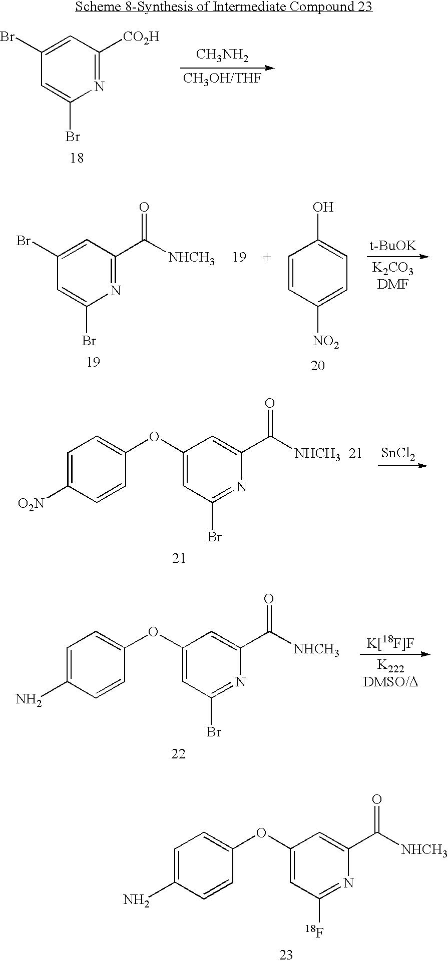

Analogue compound 1d is then prepared by coupling radiofluorinated aryl ether compound 23 with compound 17 to yield the desired product, as shown in Scheme 9.

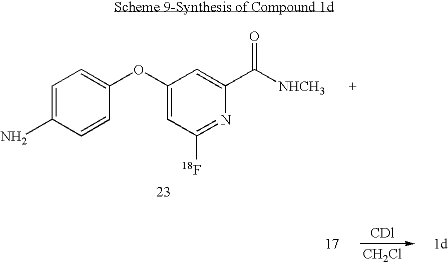

### Example 3b

### Synthesis of Radioiodinated Analogues, Compounds 1e to 1h

The radioiodinated analogues are synthesized via deiodoboronation reactions. The general synthetic scheme parallels that for the above radiofluorinations except that the radioiodine is introduced in the final step, as illustrated in Scheme 10 for the preparation of compound 1e. The chemistry shown in Schemes 10 to 13 illustrate the incorporation of iodine-124 but can be used likewise to incorporate iodine-125 and iodine-123.

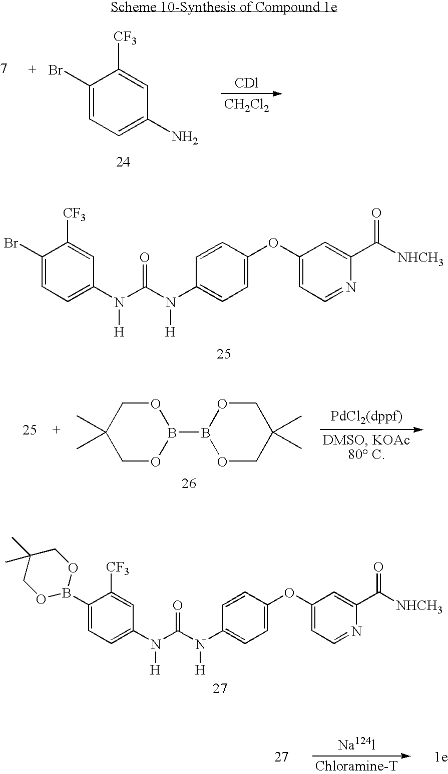

Coupling compound 7 with 4-bromo-3-trifluoromethylaniline, compound 24, in the presence of 1,1′-carbonyl imidazole generates 4-(4-[3-(4-bromo-3-trifluoromethylphenyl)-ureido]phenoxy)-pyridine-2-carboxylic acid methylamide, compound 25. Suzuki coupling of compound 25 with bis(pinacolato)diboron, compound 26, yields boronate compound 27 which is converted to compound 1e as shown.

The preparation of compound 1f involves a parallel methodology but is initiated with the 2-bromoaniline derivative, compound 28, which leads to the boronate compound 29 and, finally, to compound 1f, as shown in Scheme 11.

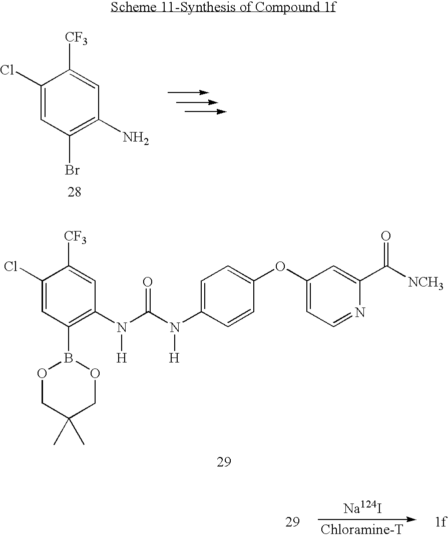

Analogue 1g can be synthesized as in Scheme 12. The iodinated intermediate compound 31 is prepared from the corresponding 2-amino-3-iodophenol, compound 30, and utilized to prepare the iodinated bis-urea compound 32 via reaction with chloroanaline compound 17. The boronate ester compound 33 is prepared by coupling compound 32 with diboron ester compound 26. Iododeboronation is then utilized to prepare compound 1g.

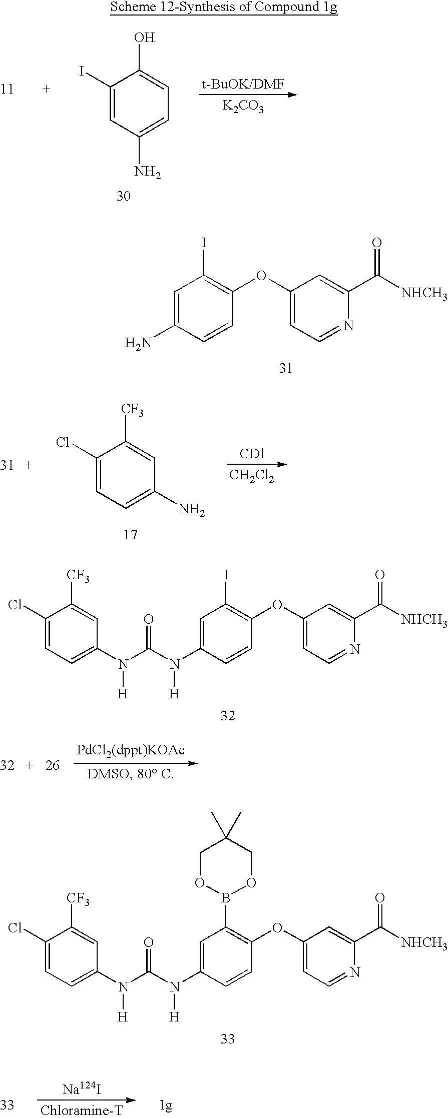

Analogue compound 1h is obtained by the preparation of aryl ether compound 34 from the corresponding 4,6-diiodopicolinic acid (which is prepared by oxidation of the corresponding 4,6-diodo-2-methylpyridine in a fashion parallel to preparation of 21 as shown in Scheme 8) which is coupled to aniline compound 17 to form compound 35, which is boronated to form compound 36. Radioiodination of compound 36 generates compound 1h, as shown in Scheme 13.

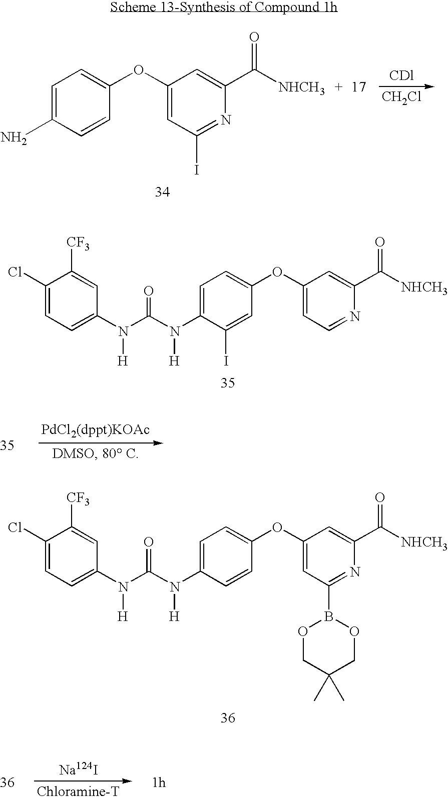

### EXAMPLE 4

The in vitro binding of the radiohalogenated analogues of BAY 43-9006 of Example 3 is verified using the human Small Cell Lung Cancer (SCLC) cell line NCI-H69 which has been shown to overexpress Raf Cells are seeded into 96-well plates (30,000 cells/well, five wells per treatment group). The analogue under study, labeled either with ¹⁸F or ¹²⁵I, is then added. Cells are pretreated with the non-radioactive analogue (1 μM) to verify the specificity of binding. Cells are incubated at 37° C. Cells are then lysed and harvested onto filters with two washes in PBS. Radioactivity bound to the filters is measured using a well counter and data obtained is analyzed for statistical significance by ANOVA and student's t-test.

The in vivo binding of the radiohalogenated analogues of BAY 43-9006 of Example 3 is verified using Balb/C mice carrying xenografts of the human SCLC cell line NIH-H69. The mice are intravenously injected with 10 μl of a solution of the radiohalogenated analogue (5-50 μCi) without or without pre-injection of the respective non-radioactive analogue (10 mg/kg). Animals, 5 per group, are sacrificed by CO₂ asphyxiation after 30, 60, or 120 minutes. Blood, lungs, liver, kidneys, muscle, heart, brain, bone, stomach, large intestine, small intestine, and tumor xenografts are harvested and weighed. Radioactivity in each sample is quantified using a well counter. Means and standard deviation of percentage of injected dose per gram (decay corrected) are calculated and data is analyzed by ANOVA and Bonferroni and Dunnett tests.

### EXAMPLE 5

Syrian Golden Hamsters are injected subcutaneously with the tobacco-specific carcinogenic nitrosamine 4-(methylnitrosamino)-1-(3-pyridyl)-1-butanone (NNK) at a dosage of 2.5 mg/100 g bodyweight three times weekly for 20 weeks. This protocol has been shown to cause the development of cancers, including Non-Small Cell Lung Cancer (NSCLC) with phenotypic features of adenocarcinoma. These NNK-induced cancers express activating point mutations in K-Ras, rendering Ras and downstream effectors, such as Raf, constitutively active with resultant hyperexpression of the downstream effectors. The NNK-induced cancers also over-express PKA, CREB and p-CREB (phosphorylated CREB). Typically, this protocol results in the development of about 10 lung tumors/hamster within 52 weeks after the start of the NNK injections with tumors of microscopic size (2 to 4 mm in diameter) present as early as 12 weeks. Control hamsters are untreated.

### EXAMPLE 6

Raf is monitored using radiohalogenated analogues of BAY 43-9006 of Example 3. Ten hamsters are imaged for each compound of interest before the start of the tumor induction protocol of Example 5, at the completion of the 20-week tumor induction protocol, and at 52 weeks after the start of the protocol.

The hamsters are anesthetized by intraperitoneal injection of 200 mg/kg ketamine and 10 mg/kg xylazine and are cannulated in the saphenous vein. The animal is then positioned in a microPET scanner with its head just below the edge of the active field of view and its lungs in the center of the field of view. A transmission scan is performed for 10 minutes. At the conclusion of the transmission scan, a 60-minute list-mode acquisition is initiated followed by a 0.5 to 1 mCi injection of the radiolabeled agent. The singles count rate from the 60-minute acquisition is analyzed to determine the exact point of injection in scanner time. Data is decay corrected using the injection time as point zero. Raw projection data is rebinned into sinograms every 10 seconds from 0 seconds to 120 seconds, then every minute to 5 minutes, then at 10 minutes, then every 10 minutes until 60 minutes. The resulting sinograms are reconstructed using both filtered backprojection and iterative reconstruction. Image quality and kinetics of the resulting dynamic PET images are analyzed to optimize the selection of time frames.

Standard region-of-interest (ROI) analysis is performed by manually contouring ROIs on the relevant transaxial image planes. In addition to tumors, ROIs are defined on normal tissues such as the spleen, heart, lung, and kidneys.

The images of the control hamsters show no areas of concentration of radioisotope except at the site of injection. In contrast, treated hamsters show concentrations of radioisotope label in the lungs and other organs. Histological and histochemical evaluation of these sites one week later (after required decay of radioactivity) confirm the presence of early premalignant lesions (formation of pseudo-ducts) and tumors and highly positive immunoreactivity to an antibody against Raf in the diseased tissues of the NNK treated hamsters.

### EXAMPLE 7

Radiohalogenated analogues of ZM 336372, an inhibitor of the Raf protein, suitable for use as tracers in PET or SPECT imaging, are produced in a similar manner to that for the production of the analogues of BAY 43-9006 as described in Example 3. The structure of ZM 336372 is shown below as Formula 2. Table 2 shows the radiohalogenated analogues of ZM 336732, compounds 37a-g that are produced.

Table 2
Radiohalogenated Derivates of ZM 336372

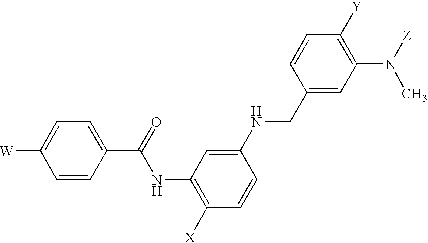

37a W = OH; X = CH_{3; Y = 4; Z =} ¹¹CH₃
37b W = OH; X = CH_{3; Y =} ¹²³I; Z = CH₃
37c W = OH; X = CH_{3; Y =} ¹⁸F; Z = CH₃
37d W = OH; X = ¹²³I; Y = H; Z = CH₃
37e W = OH; X = ¹⁸F; Y = H; Z = CH₃
37f W = ¹²³I; X = CH_{3; Y = H; Z = CH}₃
37g W = ¹⁸F; X = CH_{3; Y = H; Z = CH}₃

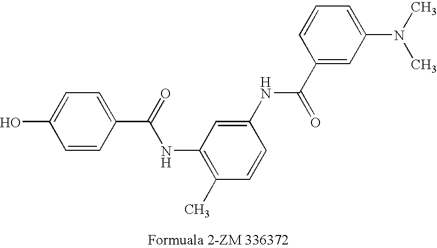

Radiohalogenated derivatives of ZM 336372 are administered to hamsters of Example 5 as described above and imaged as described in Example 6. Results are similar to those obtained using radiohalogenated derivates of BAY 43-9006.

### EXAMPLE 8

A radiohalogenated analogue of 4-cyano-3-methylisoquinoline, an inhibitor of PKA, suitable for use as a tracer in PET or SPECT imaging, is produced using either electrophic iodination. The radioanalogue, compound 38, is produced by either of the following reaction shown as Scheme 14.

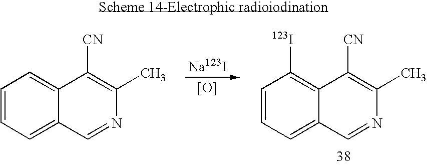

### EXAMPLE 9

Radiohalogenated analogues of Balanol, an inhibitor of the PKA protein, suitable for use as tracers in PET or SPECT imaging, are produced as described below. Table 3 shows the radiohalogenated analogues of Balanol, compounds 39a-d, that are produced.

TABLE 3

| | |
|---|---|
| | 1 |
| 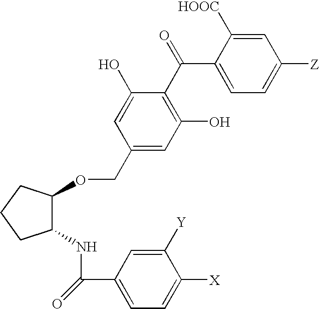 | |

39a: X = ¹²³I; Y = H; Z = H
39b: X = H; Y = ¹²³I; Z = H
39c: X = H; Y = H; Z = ¹²³ I
39d: X = H; Y = H; Z = ¹⁸F

### Example 9a

As shown in Scheme 15, 2-Aminocyclopentanol 40 is allowed to react with acid chloride 41 in the presence of dichloromethane at room temperature to generate 42 which is coupled with benzyl alcohol 43, to generate 44. The radioiodination of 44 using chloramine-T and Na¹²³I followed by removal of the benzyl group in the presence of 20% Pd(OH)2/C produces the desired product 39a.

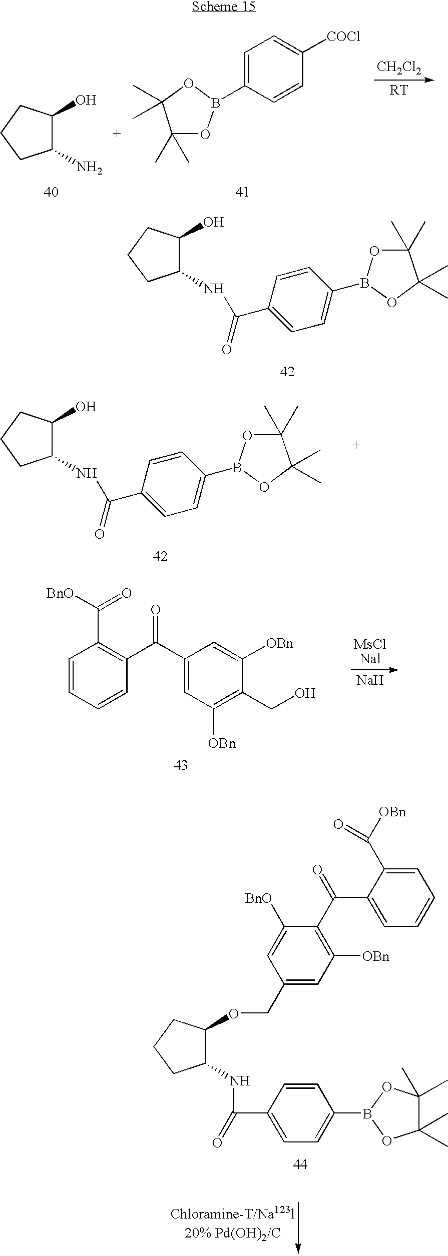

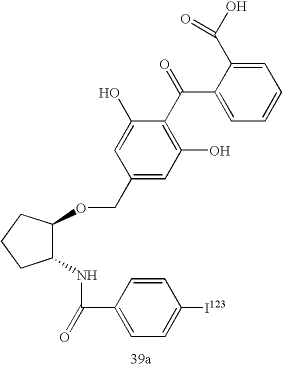

### Example 9b

As shown in Scheme 16, 2-Aminocyclopentanol 40 is allowed to react with acid chloride 45 in the presence of dichloromethane at room temperature to generate 46 which is coupled with benzyl alcohol 43 to generate 47. The radioiodination of 47 using chloramine-T and Na¹²³I followed by removal of the benzyl group in the presence of 20% Pd(OH)₂/C produces the desired product 39b.

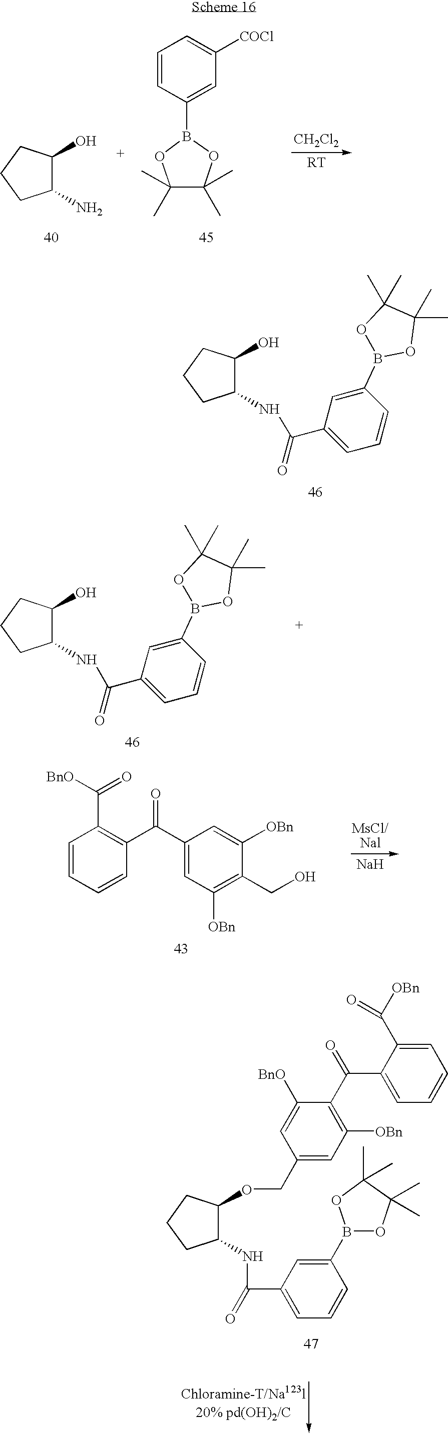

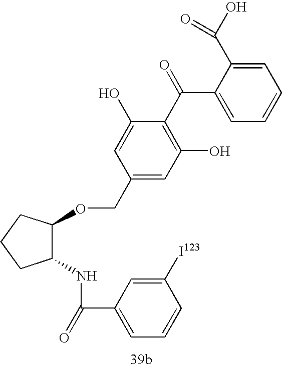

### Example 9c

As shown in Scheme 17, 2-Aminocyclopentanol 40 is allowed to react with acid chloride 48 in the presence of dichloromethane at room temperature to generate 49 which is coupled with benzyl alcohol 50, to produce 51. The radioiodination of 51 using chloramine-T and Na¹²³I followed by removal of the benzyl group in the presence of 20% Pd(OH)2/C generates the desired product 39c.

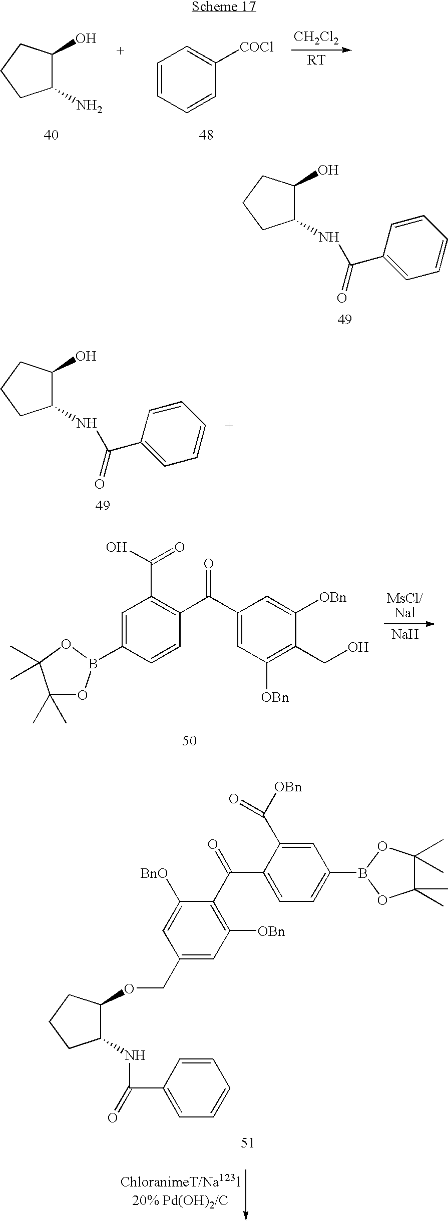

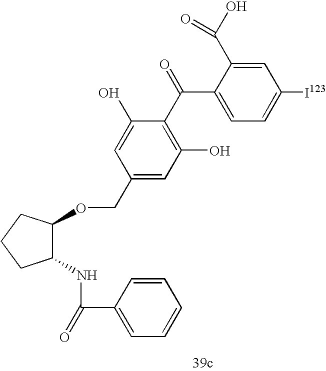

### Example 9d

As shown in Scheme 18, 2-Aminocyclopentanol 40 will be allowed to react with acid chloride 48 in the presence of dichloromethane at room temperature to generate 49¹ which will be coupled with benzyl alcohol 52 to produce 53². The radiofluorination⁴ of 53 using Kryptopix and K¹⁸F followed by removal of the benzyl group¹ in the presence of 20% Pd(OH)2/C twill generate the desired product 39d.

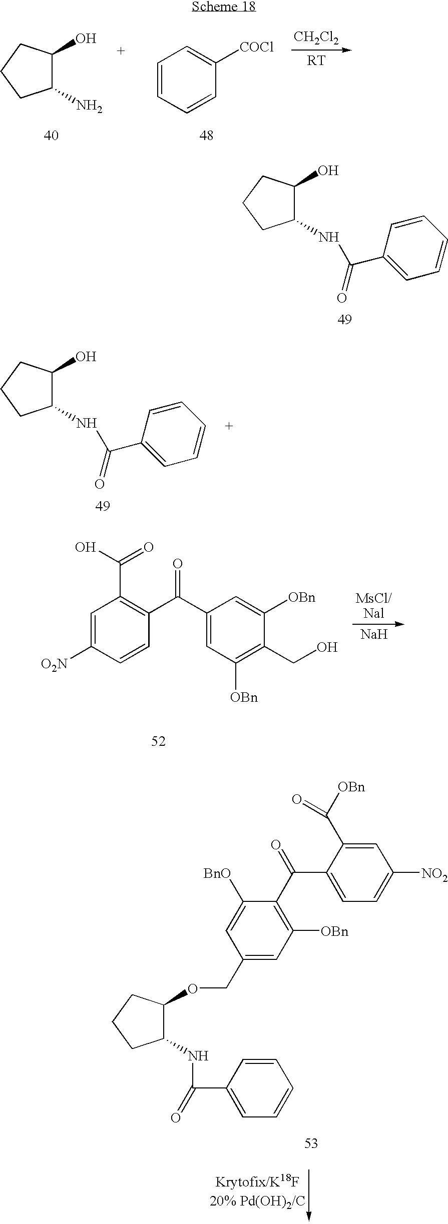

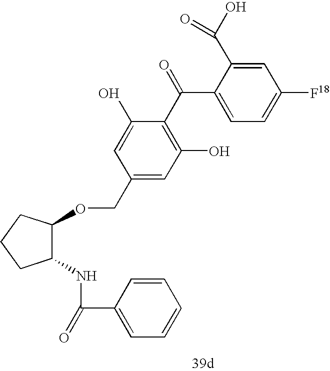

### EXAMPLE 10

Radiohalogenated derivatives of as produced by the methods described in Examples 8 and 9 are administered to hamsters of Example 5 as described above and imaged as described in Example 6. Treated hamsters show concentrations of radioisotope label in the lungs and other organs. Histological and histochemical evaluation of these sites one week later (after required decay of radioactivity) confirmed the presence of early premalignant lesions (formation of pseudo-ducts) and tumors and highly positive immunoreactivity to an antibody against PKA in the diseased tissues of the NNK treated hamsters.

### EXAMPLE 11

The following antibodies that bind to phosphorylated or unphosphorylated Ras, Raf, MAPK, ERK, PKA, PKC, CREB, and EGFR-specific tyrosine kinases are purchased from commercial suppliers. (1) Rabbit Anti-ERK (p44/42 MAP Kinase), phospho (Thr202/Tyr204) Polyclonal Antibody, Unconjugated from Covance Research Products, Inc. (Denver, Pa.); (2) Anti-Ras Antibody, Unconjugated from Cell Signaling Technology; (3) Rabbit Anti-PKA, NT Polyclonal Antibody, Unconjugated from Upstate; (4) Anti-PKC delta, Phospho (Thr505) Polyclonal Antibody from Cell Signaling Technology; (5) Anti-A-Raf Monoclonal Antibody, Unconjugated, Clone I from BD Biosciences Pharmingen; (6) Rabbit Anti-cAMP Polyclonal Antibody, Unconjugated from BioVision (Mountain View, Calif.); and (7) Rabbit Anti-MAP/ERK Kinase 1 (MEK-1) Polyclonal Antibody, Unconjugated from Covance Research Products, Inc.

The above antibodies are radiolabeled by the method of Vaidyananthan et al, Bioconjugate Chem., 12:428-438 (2001), incorporated herein by reference. Briefly, a 200 mM solution of EDC (1-ethyl-3-(3-dimethylamino-propyl)carbodiimide hydrochloride) in 100 mM MES. (2-(N-morpholino)ethanesulfonic acid) buffered saline pH 4.7 (Pierce Biotechnology, Inc.) is prepared. To 0.5 to 1.0 mCi of a radioisotope such as ¹²³I in a one-half dram vial is added 5 microliters of the EDC solution followed by 5 μliters of sulfo N-hydroxysuccinimide (20 mM in MES). The vial is vortexed and the reaction is allowed to proceed for 5 minutes at room temperature. Unreacted EDC is quenched by adding 5 μliters of 750 mM sodium acetate, and 50 liters of the antibody (about 375 mg) in pH 8.5, 0.1 M borate buffer is added. The mixture is incubated for 30 minutes at room temperature, and the labeled antibody is isolated by gel filtration chromatography over a PD10 column (Pharmacia) eluted with PBS.

Five eight week old male outbred Syrian golden hamsters are injected subcutaneously with NNK as described above in Example 5, except that the NNK injections are administered three times weekly for 10 weeks. Five age-matched controls are given identical injections with a saline vehicle without NNK. One day after the last injection, the hamsters are injected subcutaneously with an iodine-124 labeled antibody tracer. The hamsters are anesthetized by ketamine/xylazine. Each animal is positioned in a microPET scanner (microPET® P4 scanner (Concorde Microsystems, Knoxyille, Tenn.)) with its head just below edge of the active field of view and the lungs approximately in the center of the field of view. A transmission scan is then performed for 10 minutes. At the conclusion of the transmission scan, a 60-minute list-mode acquisition is initiated immediately followed by a 0.5-1 mCi injection of the radiolabeled antibody agent. The singles count rate from the 60-minute acquisition is analyzed to determine the exact point of injection in scanner time. The data is decay corrected using the injection point as time zero. The raw projection data is rebinned initially into sinograms with the following time frames: 0 sec, 10 sec, 20 sec, 30 sec, 40 sec, 50 sec, 60 sec, 70 sec, 80 sec, 90 sec, 100 sec, 110 sec, 2 min, 3 min, 4 min, 5 min, 10 min, 20 min, 30 min, 40 min, 50 min, and 60 min. The resulting sinograms are reconstructed using both filtered back projection and iterative reconstruction. The image quality and kinetics of the resulting dynamic PET images are analyzed to optimize the selection of time frames. Since the data are acquired in list mode, an arbitrary number of dynamic time sequences can be reconstructed to determine the optimal temporal sampling. CT (coaxial tomography) imaging is performed with 0.75 mm axial sampling to obtain isotropic sub-millimeter resolution. At the conclusion of the CT scan, a PET emission image is acquired as part of the standard PET/CT protocol.

The PET/CT and microPET images are co-registered using two different methods. In the first method, the fiducial markers are manually identified in both data sets to perform a point-based rigid co-registration. In the second method, automated rigid registration is performed based on mutual information. The co-registered CT images are segmented into regions of lung, soft tissue, and bone based on the histogram of Hounsfield units. The corresponding segmented pixels are assigned attenuation coefficients of 0.178, 0.095, and 0.032 cm-1 respectively. The resulting 511 keV transmission maps are then used to perform attenuation and scatter correction. Fused microPET and CT images are also generated to improve the interpretation of the microPET images.

Standard region-of-interest (ROI) analysis is performed by manually contouring ROIs on the relevant transaxial image planes. In addition to tumors, ROIs are defined on normal tissues such as the spleen, heart, lung, and kidneys. Time-activity curves (TACs) are generated for each tissue ROI type by calculating the mean and maximum radioactivity over the ROIs. Standardized uptake values (SUV) are calculated at various time points by dividing the TACs by the injected radioactivity. The ratio of tumor SUV to normal tissue SUVs will be calculated to determine the optimal time for imaging the maximum tumor to background ratio.

Animals imaged with iodine-124 also have a 30-minute static microPET scan at 24 hours post-injection to study normal tissue clearance and tumor retention.

The images of the control hamsters show no areas of concentration of radioisotope except at the site of injection. In contrast, treated hamsters show concentrations of radioisotope label in the lungs and other organs. Histological and histochemical evaluation of these sites one week later (after required decay of radioactivity) confirm the presence of early premalignant lesions (formation of pseudo-ducts) and tumors and highly positive immunoreactivity to an antibody against various members of the MAPK and GPCR pathways in diseased tissue of the NNK treated hamsters.

Further modifications, uses, and applications of the invention described herein will be apparent to those skilled in the art. It is intended that such modifications be encompassed in the claims that follow.

1. A method for diagnosing a bodily disorder in a mammalian subject, which bodily disorder is associated with an increased expression and/or increased activity of one or more members of a signal-induced protein activation cascade selected from the group consisting of the MAPK pathway and the G protein-coupled receptor (GPCR) pathway, comprising non-invasively contacting a reagent that binds to a member of said pathways to a tissue or fluid of said subject, permitting the reagent to bind to the member of said pathways that may be present in said tissue or fluid, determining the presence of said binding, and correlating said binding with the presence or severity of the bodily disorder within the subject.

2. The method of claim 1 wherein the bodily disorder is a cancer.

3. The method of claim 1 wherein the member is a member of the MAPK pathway.

4. The method of claim 1 wherein the member is a member of the GPCR pathway.

5. The method of claim 1 wherein the contacting is to a fluid that has been removed from the body of the subject.

6. The method of claim 5 wherein the fluid is blood.

7. The method of claim 1 wherein the reagent is an antibody.

8. The method of claim 1 wherein the reagent is a radiolabeled chemical compound.

9. The method of claim 1 wherein the subject is known to be suffering from a disease that is associated with increased expression of either or both of the MAPK and GPCR pathways.

10. The method of claim 1 wherein the contacting is by administering to the subject a labeled reagent that binds to said member of the pathways.

11. The method of claim 10 which further comprises permitting the labeled reagent to be distributed within the body of the subject and to bind to said member within the body of the subject and determining the presence of one or more areas within the body of the subject where the reagent has bound to the member.

12. The method of claim 10 wherein said label is a radioisotope label.

13. The method of claim 10 wherein the determining of the presence of areas within the body where the reagent has bound is by an imaging modality that produces an image of a portion or of the entire body of a subject.

14. The method of claim 13 wherein the imaging modality is nuclear imaging.

15. The method of claim 14 wherein the nuclear imaging modality is selected from the group consisting of nuclear medicine, positron emission tomography, and single photon emission computerized tomography.

16. A method for diagnosis of a disease condition in a mammalian subject comprising removing a test fluid sample from the subject, contacting the fluid sample with a reagent that binds to a member of a signal-induced protein activation cascade selected from the group consisting of the MAPK pathway and the G protein-coupled receptor (GPCR) pathway, and comparing the amount of binding of said reagent with said member in the test sample with the amount of binding that occurred in a sample removed previously from said subject or with the amount of binding that occurs in a similar sample removed from another subject not suffering from a disease condition associated with increased activity or increased expression of the MERK and/or GPCR pathways, thereby diagnosing the progression or presence of such a disease condition in the subject from which the test sample was removed.

17. The method of claim 16 wherein the fluid is blood.

18. The method of claim 16 wherein the reagent is an antibody.

19. A method for diagnosing a bodily disorder in a mammalian subject, which bodily disorder is associated with an increased expression and/or increased activity of one or more members of a signal-induced protein activation cascade selected from the group consisting of the MAPK pathway and the G protein-coupled receptor (GPCR) pathway, comprising administering to said subject a labeled reagent that binds to a member of the MAPK or the GPCR pathway, permitting the administered reagent to distribute within the body of the subject and to bind to the member of the MAPK or GPCR pathway, and determining the presence or absence of one or more areas within the body of the subject where the labeled reagent has localized, thereby diagnosing the bodily disorder.

20. The method of claim 19 wherein the label is a radioactive label.

21. The method of claim 20 wherein the radioactive label is selected from the group consisting of radioactive iodine, fluorine, bromine, nitrogen, carbon, and oxygen.

22. The method of claim 20 wherein the determination is by an imaging modality that produces an image of the body of the subject.

23. The method of claim 22 wherein the imaging modality is nuclear imaging.

24. The method of claim 23 wherein the nuclear imaging is nuclear medicine, positron emission tomography, or single photon emission computerized tomography.
